(19) United States
(12) Patent Application Publication
(10) Pub. No.: US 2022/0395649 A1
(43) Pub. Date: Dec. 15, 2022

(54) INHALER BYPASS

(71) Applicant: Iconovo AB, Lund (SE)

(72) Inventors: Orest Lastow, Torna Haellestad (SE); Henrik Svedberg, Barsebaeck (SE); Lars Arvidsson, Dalby (SE); Fredrik Gunnarsson, Soedra Sandby (SE)

(21) Appl. No.: 17/775,292

(22) PCT Filed: Nov. 13, 2020
(86) PCT No.: PCT/EP2020/082039

§ 371 Date: May 7, 2022

(30) Foreign Application Priority Data
Nov. 14, 2019 (SE) 1951314-2

(51) Int. Cl. A61M 15/00

(52) U.S. Cl. CPC A61M 15/0021; A61M 15/0065; A61M 2202/064

(57) ABSTRACT

A dry powder inhaler is disclosed. The dry powder inhaler includes a first housing member and a second housing member being rotatable in relation to one another to prepare administering a medicament dose from at least one medicament reservoir. An air inlet, a first air outlet, and a second air outlet are provided. The air inlet and the first air outlet are connected by a first air channel via a dosage mechanism configured to arrange a dose from the reservoir into the first air channel. The air inlet and the second air outlet are connected by a second air channel. An inhalation chimney is provided internally forming the proximal portion of the first air channel and comprising the first air outlet at a first end thereof.

### CROSS-REFERENCE TO RELATED APPLICATIONS

This application claims priority to International Patent Application No. PCT/EP2020/082039 filed Nov. 13, 2020, which also claims priority to Sweden Patent Application No. SE 1951314-2 filed Nov. 14, 2019, each of which is hereby incorporated by reference in its entirety.

### TECHNICAL FIELD

This invention pertains in general to the field of medicament inhalers, and more particularly to dry powder inhalers.

### BACKGROUND

Inhalers are widely used in the pharmaceutical field for treatment of respiratory and/or other diseases. Numerous drugs, medications and other substances are inhaled into the lungs using the inhalers for rapid absorption of the drug etc. in the blood stream and for local action in the lung.

The present disclosure relates to a dry powder inhaler (DPI). A dry powder inhaler releases a pre-metered, capsuled, dose or a device-metered dose of powdered medication that is inhaled through the inhaler. Inhalers with a device-metered dose of powdered medication are normally inhalers with at least one medication reservoir containing powdered medication, from which metered doses are withdrawn through the use of different dose metering arrangements, the doses then being inhaled.

Dry powder inhalers must not only produce consistent and reliable doses of the medicament it contains, but it also needs to be able to produce a certain particle size of the medicament that is being delivered, in order to achieve the desired effect. The particle size should be predominantly below 5 microns, and preferably between 1 micron and 3.3 microns.

However, such small particles are often very cohesive due to high surface energy. Agglomeration may be worsened by moisture and/or when the medication comprises more than one active substance, since the different active substances may have such properties as to form agglomerations with each other or with pharmaceutical carriers etc. Agglomeration of small particles is a problem which results in the active particles leaving the inhaler as large agglomerates.

Another problem that the developers of inhalers are faced with is that the medicament should also ideally be delivered and dispersed throughout the respiratory tract and to as large of a portion of the lungs as possible, and it is desired that the as little medicament as possible ends up in the mouth and in the upper airways in the throat of the user. The inhaler should preferably require as little effort as possible from the user while still achieving the desired effect.

As such, there is in the field of powder inhalers a constant strive to provide a dry powder inhaler device with improved dispersion of the dry powder, which facilitates delivery of the medicament throughout the lungs of the user, and which provides consistent and reliable medicament dosage.

EP0547429 A1 shows a powder inhalator and represents an example of prior art devices.

### SUMMARY

Accordingly, the present invention preferably seeks to mitigate, alleviate or eliminate one or more of the above-identified deficiencies in the art and disadvantages singly or in any combination and solves at least the above mentioned problems by providing a dry powder inhaler comprising a first housing member and a second housing member being rotatable in relation to one another in order to prepare administering of at least one medicament dose from at least one medicament reservoir of said dry powder inhaler. The inhaler further comprising at least one air inlet, a first air outlet and a second air outlet, the at least one air inlet and the first air outlet are connected by a first air channel via a dosage mechanism configured to upon relative rotation of said first and second housing members arrange at least one dose of at least one medicament from the at least one medicament reservoir into said first air channel. The at least one air inlet and the second air outlet are connected by a second air channel, and the dry powder inhaler further comprises an inhalation chimney internally forming the proximal portion of the first air channel and comprising the first air outlet at a first end thereof. The inhalation chimney is a separate part being connected to the inhaler such that axial movement of the inhalation chimney is allowed at least in relation to the first housing member. Providing an inhaler where the inhalation chimney is a separate part that is axially moveable is beneficial in terms of manufacturing and assembly. The axially moveable chimney will absorb dimensional variations that might occur in the manufacturing of the components of the inhaler, while ensuring that the desired function of the inhaler is maintained. The dimensional variations may be within the tolerances required, but could still, for prior art solutions, cause problems during assembly. Prior art inhalers could also risk being placed in a state of tension that could cause problems with noises forming when the inhaler is used or where the administering of medicament does not function as intended.

The second air outlet may be arranged surrounding the first air outlet, the second air outlet thus providing a shielding air flow around the air flow from the first air outlet which contains the medicament.

In one embodiment, the first end of the inhalation chimney is supported by the first housing member and a second end of the inhalation chimney is connectable to the dosage mechanism. The inhalation chimney thus forms a connection between the dosage mechanism and the first housing member for administering medicament, which can absorb dimensional variations in the components of the inhaler.

A plurality of support members may be provided on the first housing member providing radial support of the first end of the inhalation chimney while allowing axial movement thereof.

Between two and five, preferably three support members may be provided, being evenly spaced around the circumference of a cylindrical opening in the first housing member. The support members keep the chimney centered in the opening.

The support members may further be in the shape of protrusions arranged in the opening in the first housing member, the protrusions supporting the inhalation chimney by means of a press fit connection. The press fit connection allows the chimney to be easily mounted to the first housing member, and supported thereby with sufficient friction so that it will not move axially under its own weight. This reduces the risk of the chimney causing undesired noises or becoming incorrectly positioned in the inhaler.

The inhalation chimney may have an annular cross-sectional shape internally forming a circular first air outlet and an annular second air outlet surrounding the first air outlet. The above arrangement of the air outlets provide a central air flow which is at least to some extend shielded and surrounded by the outer annular air flow from the second air outlet. The shielded central airflow contains medicament which can, by the shielding effect from the airflow from the second air outlet, be more efficiently delivered to the desired portions of the airways and lungs of the user.

In one embodiment, the first air outlet is arranged offset below the surrounding surface of the first housing member, thus to some extent shielding the inhalation chimney from the user's mouth. This facilitates keeping undesired particles from entering the dosage mechanism.

The inhalation chimney may further comprises axially extending grooves in which the support members are configured to be arranged. The grooves improve the ability for the support members to, via the grooves, transfer a torque to the dosage mechanism during administering of medicament.

Further still, the inhalation chimney may comprise protrusions, the protrusions forming axial stops limiting the axial movement of the inhalation chimney such that the first end thereof does not protrude outside the first housing member. The protrusions are preferably arranged on the outwardly facing peripheral surface of the chimney.

Further advantageous embodiments are disclosed below and in the appended patent claims.

### BRIEF DESCRIPTION OF THE DRAWINGS

These and other aspects, features and advantages of which the invention is capable will be apparent and elucidated from the following description of embodiments of the present invention, reference being made to the accompanying drawings, in which

FIG. 1 is a cross sectional view of the inhaler along a longitudinal axis of the inhaler according to an embodiment;

FIG. 2 is a perspective view of the inhaler according to an embodiment;

FIG. 3 is a top view of the upper part of the inhaler in FIG. 1, wherein the lower portions of the inhaler are omitted for clarity;

FIG. 4 is a detail cross sectional view of the mouthpiece of the inhaler according to one embodiment; and

FIG. 5 is cross sectional view of the inhaler according to one embodiment.

### DETAILED DESCRIPTION

The following description focuses on embodiments of the present invention applicable to a medicament inhaler 100, and in particular to a dry powder drug inhaler with more than one medicament reservoir, such as two medicament reservoirs. However, it will be appreciated that the invention is not limited to this application but may be applied to many other inhalers having an inlet and an outlet, as well as a medicament reservoir.

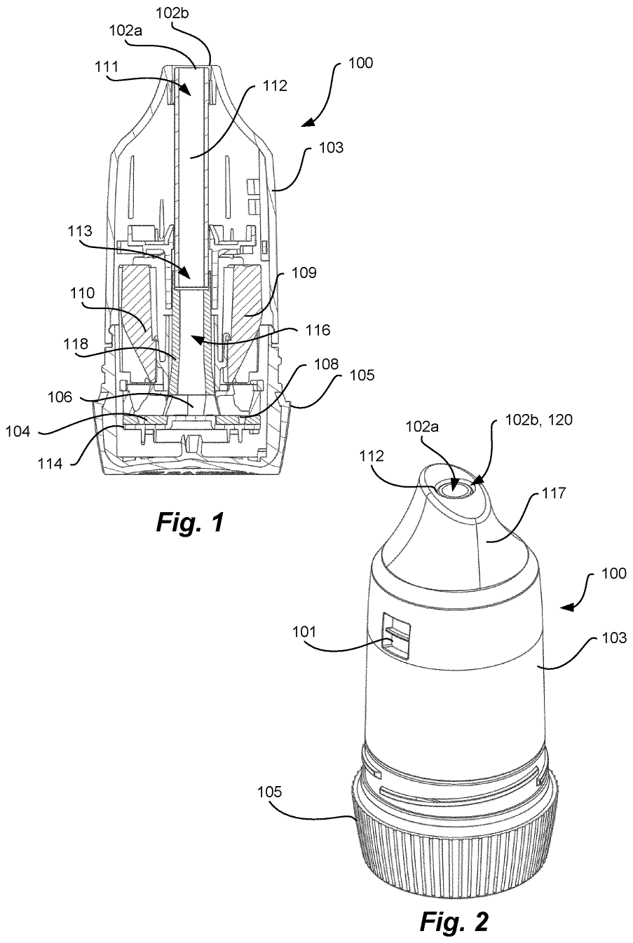

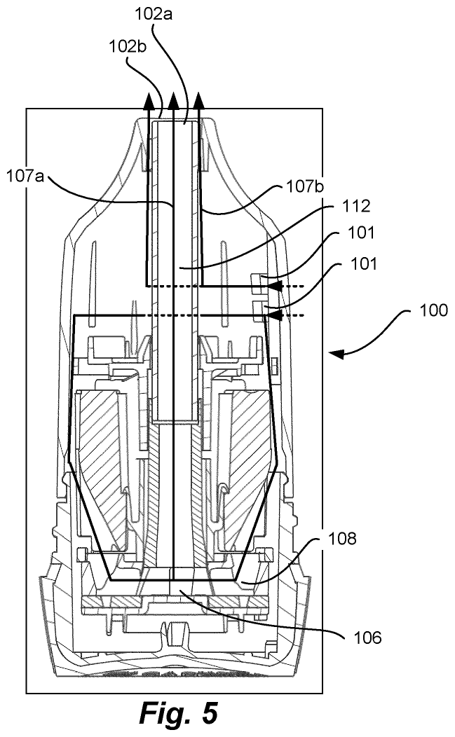

FIGS. 1 and 2 illustrate a dry powder drug inhaler 100 in a cross-sectioned and a perspective view respectively. The dry powder drug inhaler 100 comprises at least one air inlet 101 and first and second air outlets 102a, 102b. The outlets 102a, 102b are arranged in a zone of a proximal end on a mouthpiece 117 of the dry powder drug inhaler 100. The air inlet(s) 101 are connected to the first and second air outlets 102a, 102b by a respective first and second air channel 107a, 107b, which are further discussed in conjunction with FIG. 5. It is however to be realized that the distinction between the first and second air channels 107a, 107b is important as only the first air channel 107a delivers medicament, mixed with air. The second air channel 107b provides only air. Proximal in the present disclosure refers to any portion of the inhaler 100 intended to be arranged close to a user, while the opposite naturally is the case for distal. The air inlets 101 may be arranged at the periphery of the dry powder inhaler 100 in a radial position in relation to the longitudinal axis of the dry powder drug inhaler 100, such that the inlets 101 lead inhaled air transversally and radially towards the central portion of the dry powder inhaler 100.

The number of inlets and outlets 101, 102a, 102b may be different from what is disclosed in FIGS. 1 to 5. The number of inlets 101 may for example be adjusted in accordance with needs and specific inhaler design such that a number of smaller air inlets, for reducing pressure fall over the inhaler, are arranged circumferentially on the dry powder inhaler 100.

The different parts of the dry powder inhaler 100 may be manufactured in a suitable material, such as injection moldable plastics, such as thermoplastics.

The dry powder inhaler 100 comprises three major parts in the form of (i) an upper, proximal first housing member 103, preferably in the shape of a reservoir housing 103, (ii) a dosage mechanism 118 which may comprise a dose disc 104 having at least one cavity 108, a mixing and de-aggregation chamber 106 adjacent to the at least one cavity 108, and a conduit 116 extending distally from the chamber 106, and (iii) a second housing member 105, preferably in the shape of a lower distal twister 105 which in some embodiments may comprise a floor disc 114.

The first housing member 103 and the second housing member 105 cooperate so as to house the dosage mechanism 118 and optionally the floor disc 114 in between housing 103 and twister 105. An inhalation chimney 112 preferably cooperates with the conduit 116 of the dosage mechanism 118 such that the dose disc 104 may be rotated between a dose administering position and a dose collecting position when the reservoir housing 103 is rotated. The floor disc 114 is connected to the second housing member 105, i.e. the twister 105, so that the floor disc 114 only moves when relative rotation is achieved between the first housing member 103 and the second housing member 105. This may be accomplished by connecting the floor disc 114 and the second housing member 105 via interconnecting grooves and ribs, or letting the twister 105 extend longitudinally around the floor disc 114.

The floor disc 114 is located underneath dose disc 104 and extends substantially across the diameter of the dose disc 104. The floor disc 114 abuts the dose disc 104 and closes the bottom of the cavities 108 to provide support for the medicament in cavities 108 after collection of the medicament from the reservoirs 109 and 110. Thus, the floor disc 114 forms a bottom of the cavity 108, wherein said dose disc 104 rotates in relation to the floor disc 114 when the dose disc 104 is rotated between the dose collecting position and the dose administering position. Floor disc 114 moves with the second housing member 105 (i.e. the twister 105) when the twister 105 is rotated. The floor disc 114 does not rotate independently of lower twister 105. In use the dose disc 104 is rotated independently of the floor disc 114 while remaining in contact with floor disc 114. The arrangement of the dose disc 104 to the floor disc 114 allows the inhaler to be used with free flowing powder medicaments such as carrier based formulations that are not particularly susceptible to aggregation in the cavities 108. The floor disc 114 supports the free flowing powder so that cohesive forces between particles of the powder are not necessary to retain the powder in the cavity.

The arrangement further allows for a more robust inhaler that can absorb shock, shaking or other types of impact with little to no disturbance or loss of medicament from cavities 108 prior to inhalation. Even if the inhaler is vigorously shaken resulting in displacement of some medicament from a cavity 108, there is no risk of multiple dosing as the dose disc 104 is not rotated into the medicament reservoirs (again) prior to inhalation of the medicament during correct use of the inhaler. The arrangement also allows for removal of any medicament residue from the cavity 108 when the dose disc 104 is rotated over the floor disc 114. Frictional forces between the dose disc 104 and the floor disc 114 during the rotational movement result in attrition or grinding of the medicament residue that is later inhaled by the user or returned to the medicament reservoirs. These frictional forces are maximized during simultaneous rotation of the dose disc 104 and the floor disc 114 against each other by rotating the first housing member 103 and the second housing member 105 in opposite directions. This prevents accumulation of medicament residue in the cavities 108 and is most beneficial when using the inhaler with a medicament(s) susceptible to aggregation in the cavities 108.

Preferably, the rotation of the dose disc 104 has two end positions in relation to the first housing member 103 and the dosage mechanism 118 corresponding to the dose administering position and the dose collecting position in a known manner.

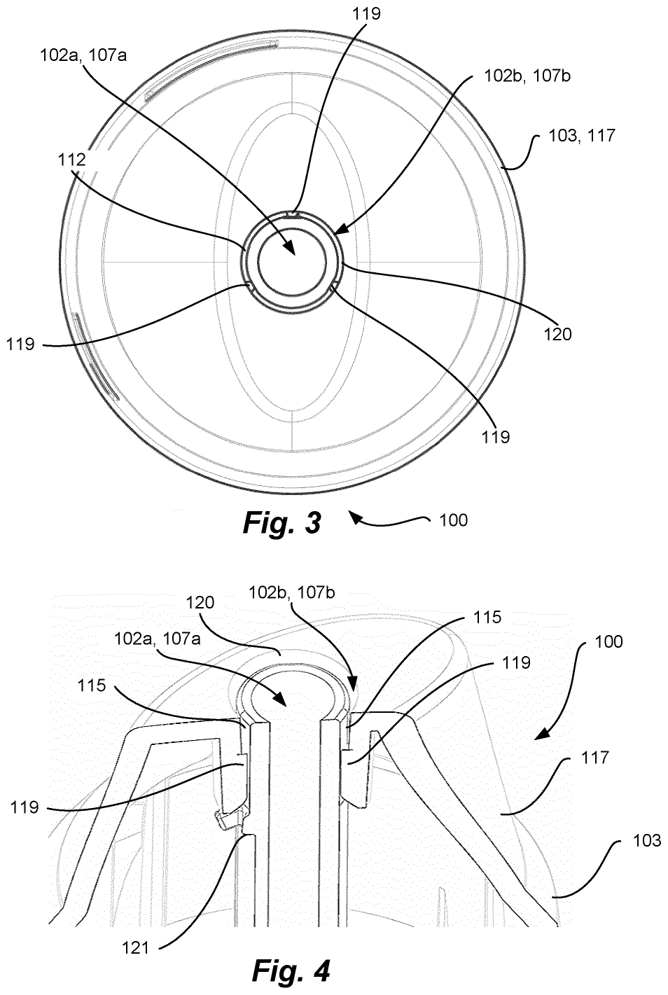

In the dose administering position, the air inlets 101 are in communication with the mixing and de-aggregation chamber 106. It is also envisioned that only some of the air inlets 101 are placed in communication with the mixing and de-aggregation chamber 106 in the dose administering position. When the dose disc 104 is rotated into a dose collecting position (not shown), the chamber 106 and the cavities 108 are rotated away from communication with the inlets 101 and the first air channel 107a. Instead, the cavities 108 are rotated into medicament reservoir 109 and medicament reservoir 110 disclosed in FIG. 3, wherein the cavities 108 may collect a medicament housed in the reservoirs 109 and 110. The medicament contained in the medicament reservoir 109 may be a medicament different from the medicament contained in the medicament reservoir 110. Due to the presence of two reservoirs 109 and 110, the inhaler 100 may deliver two substances in one inhalation, the two substances otherwise being incompatible meaning that these two substances would not be possible to be comprised in one joint reservoir. Thus, the dry powder inhaler device 100 can effectively and satisfactorily disperse two dry powders and can administer a medicament comprising two or more substances which are incompatible in a mixture or are preferably stored in separate reservoirs for other reasons.

For single medicament delivery, only one medicament reservoir 109 is required. If so, the dry powder inhaler 100 only comprises one medicament reservoir 109 or the same medicament is filled in the two medicament reservoirs 109 and 110.

It is possible to arrange the dose disc 104 and the cavities 108 thereof such that when a first set of two cavities 108 are arranged in the first air channel 107a, i.e. in a dose administering position, a second set of two cavities 108 are positioned in the medicament reservoirs 109. In this arrangement the inhaler has two medicament reservoirs, two air inlets, and one dose disc with four cavities. Additionally, the distribution of the cavities 108 on the dose disc 104 is such that the dose disc 104 may be rotated in one direction only meaning that when the second set of two cavities 108 lie in line with the first air channel 107a, the first set of cavities 108 are positioned in the medicament reservoirs 109, 110 respectively. It is also possible for the dose disc 104 to be rotated in a first direction so that cavities 108 are arranged in the first air channel 107a in a dose administering position, and then for the dose disc 104 to be rotated in the opposite direction into the dose collecting position, and thereafter again for the dose disc to be rotated in the first direction back into the dose administering position. When the dose disc 104 is rotated in a first direction into the dose administering position and the opposite direction into the dose collecting position, the dose disc 104 may have rotational stops in the dose administering position and the dose collecting position, respectively, to ensure accurate alignment of the cavities 108 in the first air channel 107a and positioning in the medicament reservoirs 109, 110 respectively.

It is also envisioned that an inhaler provided with more than two, such as three, four, five, or six, reservoirs 109, 110 with the same arrangement of inlets, outlets, air channels, dose disc, cavities etc., is within the ambit of the present invention. For example, the inhaler 100 may have three medicament reservoirs 109, three or more air inlets 101, and a dose disc with three cavities 108. Alternatively, the inhaler 100 may have four medicament reservoirs 109, four or more air inlets 101, and a dose disc with four cavities 108. It is preferred however that the inhaler 100 have two air inlets 101, being connected to the first air channel 107a and to the second air channel 107b, one first air outlet 102a for administering medicament, a second air outlet 102b, two medicament reservoirs 109, 110, and one dose disc 104 with four cavities 108.

Also other arrangements of the dosage mechanism 118 are possible, the disclosure herein is not necessarily limited to use with the dosage mechanism 118 described above.

The inhalation chimney 112 forms the proximal portion of the first air channel 107a, and thus comprises the first air outlet 102a at a first proximal end 111 of the chimney 112. The chimney 112 is formed as a separate part being connected to the first housing member 103 and a second distal end 113 thereof preferably connectable to the dosage mechanism 118, preferably to the conduit 116 thereof. The second end 113 is preferably connected to the conduit 116 by means of a rotatable connection.

Providing the chimney 112 as a separate part provides a number of benefits in simplifying manufacturing etc. It further allows the second outlet 102b optionally to be arranged surrounding the first outlet 102a, as can be seen in FIGS. 1 to 3. Such an arrangement is beneficial in that the air from the second outlet 102b, while giving advantageous pressure drop during inhalation, will also provide a shielding or cushioning effect to the central airflow which exits through the first air outlet 102a. This helps avoiding that medicament ends up in the upper airways, and also facilitates that the medicament reaches a larger portion of the lower airways and the lungs of the user.

The inhalation chimney 112 is further configured to be supported by the first housing member 103 such that it is axially moveable at least to some extent in relation to the first housing member 103. The axially moveable chimney 112 facilitates assembly of the inhaler 100, as the first housing member 103 is joined to the second housing member 105 and to the dosage mechanism 118. The permission of some axial movement in the chimney 112 will ensure that eventual variations due to manufacturing tolerances in the components of the inhaler 100 can be effectively absorbed while the chimney 112 is still securely connected to the dosage mechanism 118. The axially moveable chimney 112 further prevents stresses in the materials of the inhaler 100 to form after assembly due to said tolerance variations.

Preferably, the first housing member 103 is provided with a plurality of support members 119, providing radial support of the first end 111 of the inhalation chimney 112 as can be seen in FIGS. 3 and 4. The support members 119 may be between two and five, preferably three in total evenly spaced in a cylindrical opening 120 in the first housing member 103. The support members 119 are preferably in the shape of radial protrusions 119 arranged evenly spaced around the inwardly facing surface of the cylindrical opening 120 in the first housing member 103. The cylindrical opening 120 is arranged on the mouthpiece 117 of the inhaler, with the chimney 112 thus arranged centered in the cylindrical opening 120 by means of the support members 119. The cross-sectional shape of the chimney 112 is preferably annular, and as it is arranged centrally in the opening 120, an airflow having a circular cross section is provided through the first air outlet 102a while an annular airflow is provided through the second air outlet 102b, the latter surrounding the former.

Preferably, the support members 119 support the chimney 112 by means of a press fit connection such that chimney 112 is moveable only by applying a certain amount of force. The force required to axially move the chimney 112 should be significantly more than the weight of the chimney 112 in order to avoid noises forming during use of the inhaler 100.

In one embodiment, the outer peripheral surface of the chimney 112 comprises axially extending tracks or grooves 115, which the support members 119 are configured to be arranged in. The grooves 115 will increase the amount of torque that can be transferred by the chimney 112, preventing eventual slippage occurring when administering medicament by twisting the first and second housing members 103, 105 in relation to one another. The grooves 115 may by their respective cooperation with the support members 119 further serve to limit the axial movement of the chimney 112.

As can be seen in FIG. 4, the chimney 112 may in one embodiment comprise protrusions 121 that form axial stops, which are preferably arranged on the outwardly facing peripheral surface of the chimney 112. These prevents that the chimney 112 becomes incorrectly positioned in relation to the first proximal housing member 103. The protrusions 121 limit the axial movement of the chimney 112 in relation to the first housing member 103 by coming into contact with the support members 119. The protrusions 121 may further be designed in the form of cogs being engaged with for instance the dosage mechanism 118 or an associated dosage counter (not shown), which allow a user to keep track of the number of doses left in the reservoirs 109, 110.

The first air outlet 102a may be arranged offset below the surrounding surface on the mouthpiece 117 of the first housing member 103. Arranging the first outlet 102a below the surrounding surface on the mouthpiece 117 can improve the shielding and cushioning effect of the airflow from the second air inlet 102b. The chimney 112 itself may also be somewhat more protected from the mouth of the user, avoiding or reducing the risk that contaminants are introduced into the inhaler 100.

In FIG. 5, the inhaler 100 is seen in a cross sectioned view where the air channels 107a, 107b are illustrated schematically. Both channels could use the same air inlet(s) 101, or have separate air inlet(s) 101. Preferably, both air channels 107a, 107b use the same air inlet(s) 101. It is also to be realized that each air channel 107a, 107b could have several separate air flow paths, as shown, and is as such not limited a single air flow path. The first air channel 107a will, upon inhalation by a user, draw air from the air inlet(s) 101, which will pass around the chimney 112 and pass down around the outside of least one of the medicament reservoirs 109, 110. The first channel 107a continues past each cavity 108 that is brought in line with the first air channel 107a and then proceeds through the mixing and de-aggregation chamber 106 and onwards through the chimney 112 into the mouth of the user. The first air channel 107a exits through the first air outlet 102a.

The second air channel 107b will, simultaneously as the first air channel 107a, draw air from the air inlet(s) 101 when the user inhales through the inhaler 100. The second air channel 107b will not pass around the outside of the medicament reservoir(s) 109, 110, but instead pass unaffected without carrying medicament to the second air outlet 102b. I.e. the second air outlet 102b is not intended for administering medicament but only air, providing, in addition to the shielding effect mentioned above, an improved pressure drop during inhalation. The second inlet also reduces the flow resistance of the inhaler.

Although the present invention has been described above with reference to specific embodiments, it is not intended to be limited to the specific form set forth herein. Rather, the invention is limited only by the accompanying claims.

In the claims, the term “comprises/comprising” does not exclude the presence of other elements or steps. Furthermore, although individually listed, a plurality of means, elements or method steps may be implemented by e.g. a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second” etc. do not preclude a plurality. Reference signs in the claims are provided merely as a clarifying example and shall not be construed as limiting the scope of the claims in any way.

1. A dry powder inhaler comprising:
a first housing member and a second housing member being rotatable in relation to one another to prepare administering of at least one medicament dose from at least one medicament reservoir,
at least one air inlet, a first air outlet and a second air outlet, the at least one air inlet and the first air outlet are connected by a first air channel via a dosage mechanism configured to upon relative rotation of said first housing member and said second housing members arrange at least one dose of at least one medicament from the at least one medicament reservoir into said first air channel, wherein the at least one air inlet and the second air outlet are connected by a second air channel, and
an inhalation chimney internally defining a proximal portion of the first air channel and comprising the first air outlet at a first end thereof, wherein said inhalation chimney is a separate part being connected to the inhaler such that axial movement of said inhalation chimney is allowed at least in relation to the first housing member.

2. The dry powder inhaler according to claim 1, wherein the second air outlet is arranged surrounding the first air outlet.

3. The dry powder inhaler according to claim 1, wherein the first end of the inhalation chimney is supported by the first housing member, and wherein a second end of the inhalation chimney is connectable to the dosage mechanism.

4. The dry powder inhaler according to claim 2, wherein a plurality of support members are provided on the first housing member providing radial support of the first end of the inhalation chimney while allowing axial movement thereof.

5. The dry powder inhaler according to claim 4, wherein the plurality of support members include between two and five support members that are evenly spaced around a circumference of a cylindrical opening in the first housing member.

6. The dry powder inhaler according to claim 4, wherein the plurality of support members are structured as protrusions arranged in an opening in the first housing member, said protrusions supporting the inhalation chimney via a press fit connection.

7. The dry powder inhaler according to claim 1, wherein the inhalation chimney has an annular cross-sectional shape internally forming the first air outlet in a circular shape and the second air outlet in an annular shape surrounding the first air outlet.

8. The dry powder inhaler according to claim 1, wherein the first air outlet is arranged offset below a surrounding surface of the first housing member.

9. The dry powder inhaler according to claim 4, wherein the inhalation chimney further comprises axially extending grooves in which the plurality of support members are configured to be arranged.

10. The dry powder inhaler according to claim 1, wherein the inhalation chimney comprises protrusions, the protrusions defining axial stops limiting the axial movement of the inhalation chimney such that the first end thereof does not protrude outside the first housing member.

11. A medicament inhaler, comprising:
a first housing member and a second housing member being rotatable in relation to one another to prepare administering of at least one medicament dose from at least one medicament reservoir,
at least one air inlet, a first air outlet and a second air outlet, the at least one air inlet and the first air outlet are connected by a first air channel via a dosage mechanism configured to upon relative rotation of said first housing member and said second housing member arrange at least one dose of at least one medicament from the at least one medicament reservoir into said first air channel, wherein the at least one air inlet and the second air outlet are connected by a second air channel, and
an inhalation chimney internally defining a proximal portion of the first air channel and comprising the first air outlet at a first end thereof, wherein said inhalation chimney is a separate part being connected to the inhaler such that axial movement of said inhalation chimney is allowed at least in relation to the first housing member.

12. The medicament inhaler according to claim 11, wherein the second air outlet is arranged surrounding the first air outlet.

13. The medicament inhaler according to claim 11, wherein the first end of the inhalation chimney is supported by the first housing member, and wherein a second end of the inhalation chimney is connectable to the dosage mechanism.

14. The medicament inhaler according to claim 12, further comprising a plurality of support members are provided on the first housing member providing radial support of the first end of the inhalation chimney and allowing axial movement thereof.

15. The medicament inhaler according to claim 14, wherein the plurality of support members are arranged evenly spaced around a circumference of a cylindrical opening in the first housing member.

16. The medicament inhaler according to claim 14, wherein the plurality of support members are structured as protrusions arranged in an opening in the first housing member, said protrusions supporting the inhalation chimney via a press fit connection.

17. The medicament inhaler according to claim 14, wherein the inhalation chimney further comprises axially extending grooves in which the plurality of support members are configured to be arranged.

18. The medicament inhaler according to claim 11, wherein the inhalation chimney has an annular cross-sectional shape internally forming the first air outlet in a circular shape and the second air outlet in an annular shape surrounding the first air outlet.

19. The medicament inhaler according to claim 11, wherein the first air outlet is arranged offset below a surrounding surface of the first housing member.

20. The medicament inhaler according to claim 11, wherein the inhalation chimney comprises protrusions, the protrusions defining axial stops limiting the axial movement of the inhalation chimney such that the first end thereof does not protrude outside the first housing member.
